(19) United States
(12) Patent Application Publication
(10) Pub. No.: US 2024/0225889 A9
(43) Pub. Date: Jul. 11, 2024

(54) Female Universal Condom Device

(71) Applicants: Nora Nunez, San Bernardino, CA (US); Rosa Nunez-Mejia, La Verne, CA (US)

(72) Inventors: Nora Nunez, San Bernardino, CA (US); Rosa Nunez-Mejia, La Verne, CA (US)

(21) Appl. No.: 18/302,571

(22) Filed: Apr. 18, 2023

Related U.S. Application Data
(60) Provisional application No. 63/417,461, filed on Oct. 19, 2022.

(51) Int. Cl. A61F 6/06; A61F 6/04

(52) U.S. Cl. CPC A61F 6/065; A61F 6/04; A61F 2006/041; A61F 2006/049

(57) ABSTRACT

The present invention relates to a novel female condom device. The device is a form of contraception designed to keep women safe during sexual intercourse. The device comprises a body component comprised of a dental dam component and male condom component. Typically, the dental dam component would be a flat, thin sheet of latex or nitrile in a rectangular shape but can be any suitable size/shape. The underside of the body component of the device would comprise an adhesive to keep the device in place during sex, minimizing the transfer of bodily fluids. In position, the flat adhesive dental dam component remains outside the vagina, while the male condom component extends into the vaginal walls, as the male penis is inserted for sexual intercourse. Once in place, the device will reduce transmission of STI and STD for women by a significant percent while also helping prevent or reduce pregnancy.

### CROSS-REFERENCE TO RELATED APPLICATION

The present application claims priority to, and the benefit of, U.S. Provisional Application No. 63/417,461, which was filed on Oct. 19, 2022, and is incorporated herein by reference in its entirety.

### FIELD OF THE INVENTION

The present invention relates generally to the field of female condom devices. More specifically, the present invention relates to a universal female condom capable of reducing the risk of STI (sexually transmitted infections) and STD (sexually transmitted diseases) spread and pregnancy during sexual intercourse. Accordingly, the present disclosure makes specific reference thereto. Nonetheless, it is to be appreciated that aspects of the present invention are also equally applicable to other like applications, devices, and methods of manufacture. It should be noted that while the invention will be described with regard to heterosexual intercourse; it may be appreciated that it can be used equally effectively in homosexual intercourse to reduce disease transmission.

### BACKGROUND

By way of background, this invention relates to improvements in female condom devices. Generally, sexual intercourse with unfamiliar and/or multiple partners can lead to increased risk of pregnancy, STI, and STD. Male condoms may not be effective enough to fully protect women against STI/STD. Further, female condoms can be uncomfortable and unpleasurable to use in their current format due to lack of design options.

Furthermore, complications associated with intrauterine devices and birth control pills have mitigated against their use in most cases. It is generally accepted as fact that the condom provides the best protection from venereal disease and HIV virus, except for exercising sexual abstinence. However, condoms, as known in the prior art, are known to suffer some drawbacks. Mainly, in that they interfere with the normal progression of intercourse, which prevents some males from using them. Moreover, it is known that women are more vulnerable to AIDS and other sexually transmitted diseases than men. Further, there is no device known in the prior art that prevents conception and reduces the risk of disease and can be used solely by the female prior to intercourse. Thus, the use of a female condom gives women greater freedom and agency to negotiate sexual relations with reduced risks.

Accordingly, a need remains for a female condom that reduces the risk associated with penetration outside the female condom during coitus. For a female condom to be an effective barrier to the transmission of the AIDS virus or other viruses and microbes, it is essential that, even during repeated contacts, the penis is unable to penetrate the vagina outside the condom. That is why it is advantageous to provide a female condom which provides an effective barrier to the transmission of diseases since contact between the bodily fluids of the man and the woman is prevented.

Therefore, there exists a long-felt need in the art for a female condom device that provides users with a universal female condom capable of reducing the risk of STI and STD spread and pregnancy during sexual intercourse. There is also a long-felt need in the art for a female condom device that features a strong and durable, but comfortable, skin-safe adhesive on the back side to secure the condom device over the vagina and vulva. Further, there is a long-felt need in the art for a female condom device that includes a male condom attached to the flat, dental dam structure of the condom that accommodates the male penis to prevent any skin-to-skin contact of the genitals while having sex. Moreover, there is a long-felt need in the art for a device that minimizes the transfer of bodily fluids to help reduce the risk of STI and STD transmission without decreasing pleasure for men and women during sexual intercourse. Further, there is a long-felt need in the art for a female condom device that is configured to keep women safer during sexual intercourse. Finally, there is a long-felt need in the art for a female condom device that can be constructed of various shapes and sizes to accommodate various female vaginas and vulvas.

The subject matter disclosed and claimed herein, in one embodiment thereof, comprises a female condom device. The device is a form of contraception designed to keep women safer during sexual intercourse. The device comprises a body component comprised of a dental dam component and a male condom component. Typically, the dental dam component would be a flat, thin sheet of latex or nitrile in a rectangular shape but can be any suitable size and shape. The underside of the body component of the device would comprise an adhesive to keep the device in place during sex, minimizing the transfer of bodily fluids. In position, the flat adhesive dental dam component remains outside the vagina, while the male condom component extends into the vaginal walls, as the male penis is inserted for sexual intercourse. Once in place, the device will reduce transmission of STI and STD for women by a significant percent while also helping prevent pregnancy.

Accordingly, the adhesive of the dental dam component sticks securely to the user's skin, covering the vaginal area from the pubic bone down to the perineum. The female condom device can be constructed using both latex or non-latex materials to accommodate all personal and medical needs and preferences.

In this manner, the female condom device of the present invention accomplishes all of the foregoing objectives and provides users with a device that provides maximum comfort and protection for a user. The device is a form of contraception that is placed on a user's vagina and vulva to reduce the transmission of STI and STD. The device can be manufactured of latex or nitrile and conforms to the shape of a user's vagina.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a female condom device. The device is a form of contraception designed to keep women safer during sexual intercourse. The device comprises a body component comprised of a dental dam component and a male condom component. In position, the flat adhesive dental dam component remains outside the vagina, while the male condom component extends into the vaginal walls, as the male penis is inserted for sexual intercourse. Once in place, the device will reduce transmission of STI and STD for women by a significant percent while also helping prevent pregnancy.

The present invention generally comprises a female condom device for use in preventing or reducing both conception and transmission of venereal diseases. A salient feature of the invention is that it is designed to be used by a female prior to intercourse, rather than by the male as is known in the prior art. In one embodiment, the basic configuration of the present invention comprises a body component which includes a dental dam component and a male condom component. The male condom device is formed of a generally cylindrical tube. The tube includes a distal end having an integrally formed closure of generally hemispherical or smoothly rounded configuration, and a proximal end that is open. The proximal end opening allows for insertion of a male penis during sexual intercourse. Thus, the male condom component extends into the vaginal walls of a user during use.

In one embodiment, a generally form-retaining applicator is received within the male condom component only for purposes of insertion of the male condom component into the vagina of the female. The applicator in this embodiment comprises a solid rod or hollow tubular rod of polymer or plastic material having sufficient rigidity to be urged into the vaginal opening. The interior of the male condom component is provided with a lubricant to release the applicator rod and permit its withdrawal therefrom after insertion of the male condom component. Thus, the male condom component forms a closed lining of the vaginal walls with the proximal end of the male condom component extending outwardly of the vagina, to prevent epidermal genital contact during intercourse, and to prevent the release of semen within the vagina.

A salient feature of the female condom device of the present invention is that it is a positive step that a woman may take prior to intercourse to assure contraception and prevent disease transmission without requiring the participation or acquiescence of the male partner. Further, due to the fact that the female condom device may be inserted prior to intercourse, it does not necessarily comprise any interruption or interference with the progression of sexual intercourse.

In a further embodiment of the female condom device, the proximal end portion of the male condom component is secured to the dental dam component, which includes a rectangular-shaped portion that flares outwardly away from the proximal end to cover a user's vagina. Typically, the rectangular-shaped portion would be a flat, thin sheet of latex or nitrile in a rectangular shape but can be any suitable size and shape. The rectangular-shaped portion is larger in diameter than both the male condom component and the nominal vaginal opening. As the male condom component is to be inserted into the user's vagina forming a barrier between the male penis and the vaginal wall, the rectangular-shaped portion remains external to the vagina forming a barrier between the male penis and the vagina, as well as the adjacent perineal and vulva areas. Thus, the dental dam component acts to cover the vulva and vaginal area and extends from the user's pubic bone down to the perineum. The extended barrier created by the dental dam component is particularly useful in preventing or reducing disease transmission, such as STI and STD.

In one embodiment, the female condom device is manufactured from a resilient, biologically compatible material, such as natural rubber latex (NRL), polyethylene, polyurethane, latex rubber, nitrile, a synthetic equivalent, and derivatives and mixtures of these materials. Further, the female condom device may be lubricated inside and/or outside with a lubricant sufficient to improve the comfort.

In one embodiment, the underside of the dental dam component comprises an adhesive or adhesive layer which secures to a user's skin to keep the device in place during sex, minimizing the transfer of bodily fluids. The adhesive can be an adhesive section, multiple adhesive sections, or an entire adhesive layer positioned on the underside of the dental dam component. The adhesive can be any suitable adhesive as is known in the art, such as a strong, durable but comfortable, skin-safe adhesive, as is known in the art. Further, the adhesive typically comprises a peelable protective strip which is then applied to the adhesive. When the device is applied to a user, the strip is peeled off exposing the adhesive layer. The body component is then pressed to a user's vaginal area, sealing the vaginal area and preventing skin-to-skin contact of the genitals during sexual intercourse. Thus, the use of the adhesive retains the female condom device in place on the vagina area during and after intercourse. However, the female condom device can be easily removed by manually grasping the dental dam component and pulling the device from the vagina, the male condom component collapsing during withdrawal.

Accordingly, the female condom device also offers increased protection against the transmission of sexually transmitted diseases as well as efficient protection against unintentional pregnancies. The body component covers the vaginal and vulva area and reduces the risk of penetration outside the female condom device, which makes the female condom device safer than a standard male condom. The female condom device is held in position on the vagina during coitus via the adhesive.

In one embodiment, the thickness of the body component and the male condom component may vary, it being understood that greater thicknesses of the body component and the male condom component reduce sensitivity during coitus. Typical thickness for the body component and the male condom component is between 20 and 100 micrometers.

Of course, the thickness of the body component must be such that the resistance to mechanical stresses of the female condom device established by standards in force in different countries is guaranteed. The dimensions of the female condom device are such that they allow movement of the penis relative to the female condom device during sexual intercourse.

It may be appreciated that during insertion of the male condom component by an applicator or a male penis, etc., the tensile force applied axially to the male condom component by the applicator or male penis causes the male condom component to elongate elastically and constrict diametrically. When the applicator or penis is withdrawn, the elastic memory causes the male condom component to expand against the vaginal wall to retain the male condom component within the vaginal walls.

In one embodiment, the male condom component comprises a diametrical expansion to prevent the male condom component from coupling to the male penis and being pulled out of the vagina during intercourse. Specifically, the generally cylindrical tubular portion of the male condom component comprises at least one pleat or a plurality of pleats extending longitudinally there along. After initiation of intercourse, if the diameter of the penis is greater than the diameter of the male condom component, the pleat (or pleats) will unfold to accommodate the larger penis and assure that the male condom component will not engage and be retained on the penis. Thus, the male condom component will remain in the vagina, to be removed manually after intercourse.

In one embodiment, the female condom device may be produced in different lengths, i.e., the length of the body component may be different depending on the size of the user's vagina, so that for each user, the body component is maintained in contact with the vulva. In general, the width of the body component lies between 1 and 4 cm and preferably between land 2 cm. Of course, the width of the body component need not necessarily be uniform over the whole of its perimeter.

In one embodiment, the female condom device is rolled to reduce the length of the device for purposes of packaging. Also, it may be appreciated that the device can be unrolled from the package to a length sufficient for the anatomy of the user, so that one size female condom device may accommodate most users. Furthermore, the rolled-up portion remaining external to the vagina is unnoticeable, and the ability to take up surplus length at the exterior avoids awkward amounts of condom material in the female genital region.

In use, manual tension is applied to the distal end of the package to open the package. Thereafter the female condom device may be removed from the package and unrolled to define a suitable length for the female condom device. The peel strip is removed from the adhesive and the female condom device is then placed over the vagina, and the male condom component inserted into the vaginal opening. Furthermore, the insertion of the male condom component in the vagina prior to intercourse warms the male condom component to body temperature prior to coitus, so that the chilling effect from cold temperature of prior art male condoms is avoided. Typically, the female condom device may be put in place just before coitus or even well before. Further, unlike the standard male condom, it is unnecessary to remove the female condom device immediately after ejaculation.

It should be noted that the present invention has been described with regard to heterosexual intercourse; it may be appreciated that it can be used equally effectively in homosexual intercourse to prevent disease transmission.

In yet another embodiment, the female condom device comprises a plurality of indicia.

In yet another embodiment, a method of protecting a female user during sexual intercourse is disclosed. The method includes the steps of providing a female condom device comprising a body component with a dental dam component and a male condom component. The method also comprises removing the underside adhesive layer of the body component. Further, the method comprises positioning the body component on a user's vaginal area from the pubic bone down to the perineum, wherein the underside adhesive layer sticks securely to the user's skin. The method also comprises extending the male condom component of the device into the vaginal walls, as the male penis is inserted for sexual intercourse. Finally, the method comprises removing and disposing of the device after sexual intercourse.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the female condom device of the present invention showing the female condom device removed from the package in accordance with the disclosed architecture;

FIG. 2 illustrates a front perspective view of one embodiment of the female condom device of the present invention showing how the adhesive layer is removed from the device in accordance with the disclosed architecture;

FIG. 3 illustrates a front perspective view of one embodiment of the female condom device of the present invention showing how the device is unrolled for use in accordance with the disclosed architecture;

FIG. 4 illustrates a front perspective view of one embodiment of the female condom device of the present invention showing how the male condom component expands to accommodate male genitalia in accordance with the disclosed architecture;

FIG. 5 illustrates a front perspective view of one embodiment of the package for the female condom device of the present invention showing how the package is opened in accordance with the disclosed architecture; and

FIG. 6 illustrates a flowchart showing the method of protecting a female user during sexual intercourse in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a female condom device that provides users with a universal female condom capable of reducing the risk of STI and STD spread and pregnancy during sexual intercourse. There is also a long-felt need in the art for a female condom device that features a strong and durable, but comfortable, skin-safe adhesive on the back side to secure the condom device over the vagina and vulva. Further, there is a long-felt need in the art for a female condom device that includes a male condom attached to the flat, dental dam structure of the condom that accommodates the male penis to prevent any skin-to-skin contact of the genitals while having sex. Moreover, there is a long-felt need in the art for a device that minimizes the transfer of bodily fluids to help reduce the risk of STI and STD transmission without decreasing pleasure for men and women during sexual intercourse. Further, there is a long-felt need in the art for a female condom device that is configured to keep women safer during sexual intercourse. Finally, there is a long-felt need in the art for a female condom device that can be constructed of various shapes and sizes to accommodate various female vaginas and vulvas.

The present invention, in one exemplary embodiment, is a novel female condom device. The device comprises a body component comprised of a dental dam component and a male condom component. Typically, the dental dam component would be a flat, thin sheet of latex or nitrile in a rectangular shape but can be any suitable size and shape. The underside of the body component of the device would comprise an adhesive to keep the device in place during sex, minimizing the transfer of bodily fluids. In position, the flat adhesive dental dam component remains outside the vagina, while the male condom component extends into the vaginal walls as the male penis is inserted for sexual intercourse. Once in place, the device will reduce transmission of STI and STD for women by a significant percent while also helping prevent pregnancy.

The present invention also includes a novel method of protecting a female user during sexual intercourse. The method includes the steps of providing a female condom device comprising a body component with a dental dam component and a male condom component. The method also comprises removing the underside adhesive layer of the body component. Further, the method comprises positioning the body component on a user's vaginal area from the pubic bone down to the perineum, wherein the underside adhesive layer sticks securely to the user's skin. The method also comprises extending the male condom component of the device into the vaginal walls as the male penis is inserted for sexual intercourse. Finally, the method comprises removing and disposing of the device after sexual intercourse.

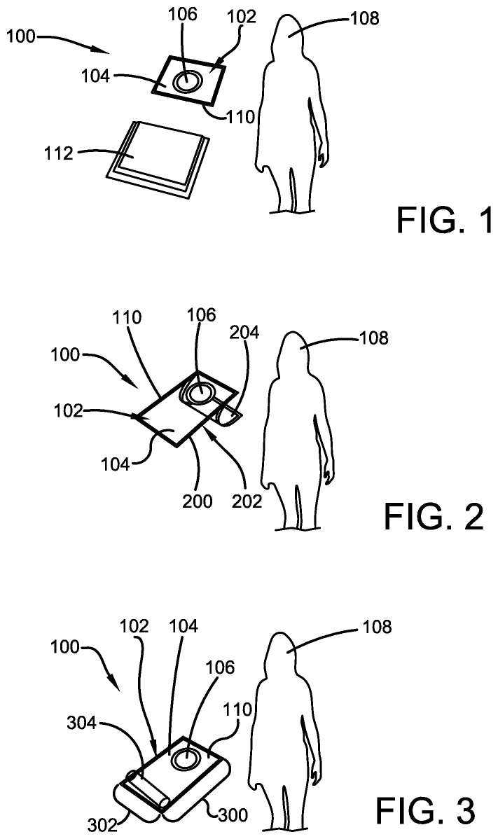

Referring initially to the drawings, FIG. 1 illustrates a perspective view of one embodiment of the female condom device 100 of the present invention. In the present embodiment, the female condom device 100 is an improved female condom device 100 that is configured to keep women safe during sexual intercourse. Specifically, the female condom device 100 comprises a body component 102 with a dental dam component 104 and a male condom component 106. In position, the dental dam component 104 remains outside the vagina, while the male condom component 106 extends into the vaginal walls as the male penis is inserted for sexual intercourse. Once in position, the device 100 is designed to reduce transmission of STI and STD for women 108 by a significant percent while also helping prevent pregnancy.

Accordingly, the present invention generally comprises a female condom device 100 for use in preventing or reducing both contraception and transmission of venereal diseases. A salient feature of the invention is that it is designed to be used by a female 108 prior to intercourse, rather than by the male as is known in the prior art. In one embodiment, the basic configuration of the present invention comprises a body component 102 which includes a dental dam component 104 and a male condom component 106. The male condom device 106 is formed of a generally cylindrical tube 400. The tube 400 includes a distal end 402 having an integrally formed closure 404 of generally hemispherical or smoothly rounded configuration, and a proximal end 406 that is open. The proximal end 406 opening allows for insertion of a male penis during sexual intercourse. Thus, the male condom component 106 extends into the vaginal walls of a user 108 during use.

In one embodiment, instead of a male penis, a generally form-retaining applicator 408 is received within the male condom component 106 only for purposes of insertion of the male condom component 106 into the vagina of the female 108. The applicator 408, in this embodiment, comprises a solid rod or hollow tubular rod of polymer or plastic material having sufficient rigidity to be urged into the vaginal opening. The interior of the male condom component 106 is provided with a lubricant (not shown) to release the applicator rod 408 and permit its withdrawal therefrom after insertion of the male condom component 106. Thus, the male condom component 106 forms a closed lining of the vaginal walls, with the proximal end 406 of the male condom component 106 extending outwardly of the vagina to prevent epidermal genital contact during intercourse and to prevent the release of semen within the vagina.

A salient feature of the female condom device 100 of the present invention is that it is a positive step that a woman 108 may take prior to intercourse to assure contraception and prevent disease transmission, without requiring the participation or acquiescence of the male partner. Further, due to the fact that the female condom device 100 may be inserted prior to intercourse, it does not necessarily comprise any interruption or interference with sexual intercourse.

A further embodiment of the female condom device 100 wherein the proximal end portion 406 of the male condom component 106 is secured to the dental dam component 104, which includes a rectangular-shaped portion 110 that flares outwardly away from the proximal end 406 to cover a user's vagina. Typically, the rectangular-shaped portion 110 would be a flat, thin sheet of latex or nitrile in a rectangular shape but can be any suitable size and shape, as is known in the art. The rectangular-shaped portion 110 is larger in diameter than both the male condom component 106 and larger than the nominal vaginal opening. As the male condom component 106 is to be inserted into the user's vagina forming a barrier between the male penis and the vaginal wall, the rectangular-shaped portion 110 remains external to the vagina forming a barrier between the male penis and the vagina, as well as the adjacent perineal and vulva areas. Thus, the dental dam component 104 acts to cover the vaginal area and extends from the user's pubic bone down to the perineum. The extended barrier created by the dental dam component 104 is particularly useful in preventing or reducing disease transmission, such as STI and STD.

As shown in FIG. 2, the female condom device 100 is manufactured from a resilient, biologically compatible material, such as natural rubber latex (NRL), polyethylene, polyurethane, latex rubber, nitrile, a synthetic equivalent, and derivatives and mixtures of these materials. Further, the female condom device 100 may be lubricated inside and/or outside with a lubricant sufficient to improve the comfort.

In one embodiment, the underside 200 of the dental dam component 104 comprises an adhesive or adhesive layer 202 which secures to a user's skin to keep the device 100 in place during sex, minimizing the transfer of bodily fluids. The adhesive 202 can be an adhesive section, multiple adhesive sections, or an entire adhesive layer positioned on the underside 200 of the dental dam component 104. The adhesive 202 can be any suitable adhesive as is known in the art, such as a strong, durable but comfortable, skin-safe adhesive, as is known in the art. Further, the adhesive 202 typically comprises a peelable protective strip 204, which is then applied to the adhesive 202. When the device 100 is applied to a user 108, the strip 204 is peeled off, exposing the adhesive layer 202. The body component 102 is then pressed to a user's vaginal area, sealing the vaginal area and preventing skin-to-skin contact of the genitals during sexual intercourse. Thus, the use of the adhesive 202 retains the female condom device 100 in place on the vaginal area during and after intercourse. However, the female condom device 100 can be easily removed by manually grasping the dental dam component 104 and pulling the device 100 from the vagina, the male condom component 106 collapsing during withdrawal.

As shown in FIG. 3, the female condom device 100 also offers increased protection against the transmission of sexually transmitted diseases as well as efficient protection against unintentional pregnancies. The body component 102 covers the vaginal and vulva area and reduces the risk associated with penetration outside the female condom device 100, which makes the female condom device 100 safer than a standard male condom. The female condom device 100 is held in position on the vagina during coitus via the adhesive 202.

In one embodiment, the thickness of the body component 102 and the male condom component 106 may vary, it being understood that greater thicknesses of the body component 102 and the male condom component 106 reduces sensitivity during coitus. Typical thickness for the body component 102 and the male condom component 106 is between 20 and 100 micrometers.

Of course, the thickness of the body component 102 must be such that the resistance to mechanical stresses of the female condom device 100 established by standards in force in different countries is guaranteed. The dimensions of the female condom device 100 are such that they allow movement of the penis relative to the female condom device 100 during sexual intercourse.

In one embodiment, the female condom device 100 may be produced in different lengths, i.e., the length 300 of the body component 102 may be different depending on the size of the user's vagina, so that for each user 108 the body component 102 is maintained in contact with the vulva. In general, the width 302 of the body component 102 lies between 1 and 4 cm and preferably between land 2 cm. Of course, the width 302 of the body component 102 need not necessarily be uniform over the whole of its perimeter.

In another embodiment, the female condom device 100 is rolled to reduce the length of the device 100 for purposes of packaging. Also, it may be appreciated that the device 100 can be unrolled from the package 112 to a length sufficient for the anatomy of the user 108 so that one size female condom device 100 may accommodate most users 108. Furthermore, the rolled-up portion 304 remaining external to the vagina is unnoticeable and the ability to take up surplus length at the exterior avoids awkward amounts of condom material in the female genital region.

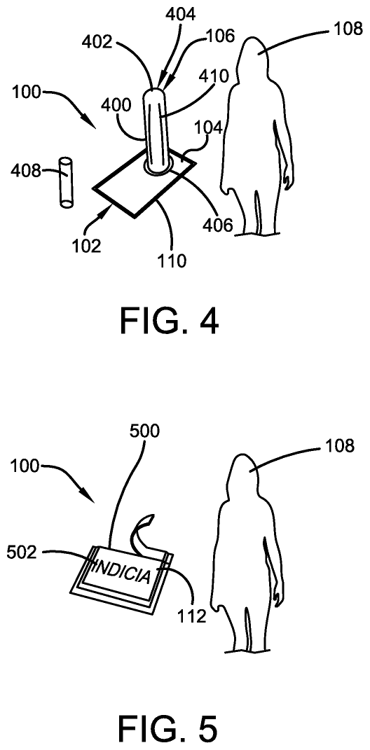

As shown in FIG. 4, it may be appreciated that during insertion of the male condom component 106 by an applicator 408 or a male penis, etc., the tensile force applied axially to the male condom component 106 by the applicator 408 or male penis causes the male condom component 106 to elongate elastically and constrict diametrically. When the applicator 408 or penis is withdrawn, the elastic memory causes the male condom component 106 to expand against the vaginal wall to retain the male condom component 106 within the vaginal walls.

In one embodiment, the male condom component 106 comprises a diametrical expansion to prevent the male condom component 106 from coupling to the male penis and being pulled out of the vagina during intercourse. Specifically, the generally cylindrical tubular portion 400 of the male condom component 106 comprises at least one pleat or a plurality of pleats 410 extending longitudinally there along. After initiation of intercourse, if the diameter of the penis is greater than the diameter of the male condom component 106, the pleat (or pleats) 410 will unfold to accommodate the larger penis and assure that the male condom component 106 will not engage and be retained on the penis. Thus, the male condom component 106 will remain in the vagina to be removed manually after intercourse.

As shown in FIG. 5, in use, manual tension is applied to the distal end 500 of the package 112 to open the package 112. Thereafter the female condom device 100 may be removed from the package 112 and unrolled to define a suitable length 300 for the female condom device 100. The peel strip 204 is removed from the adhesive 202 and the female condom device 100 is then inserted on the vagina, and the male condom component 106 inserted into the vaginal opening. Furthermore, the insertion of the male condom component 106 in the vagina prior to intercourse warms the male condom component 106 to body temperature prior to coitus, so that the chilling effect from cold temperature of prior art male condoms is avoided. Typically, the female condom device 100 may be put in place just before coitus or even well before. Further, unlike the standard male condom, it is unnecessary to remove the female condom device 100 immediately after ejaculation.

Furthermore, the female condom device 100 comprises a plurality of indicia 502. The body component 102 or the package 112 of the device 100 may include advertising, trademark, or other letters, designs, or characters, printed, painted, stamped, or integrated into the body component 102 or the package 112, or any other indicia 502 as is known in the art. Specifically, any suitable indicia 502 as is known in the art can be included, such as, but not limited to, patterns, logos, emblems, images, symbols, designs, letters, words, characters, animals, advertisements, brands, etc., that may or may not be sexual, safety, or brand related.

It should be noted that the present invention has been described with regard to heterosexual intercourse; it may be appreciated that it can be used equally effectively in homosexual intercourse to prevent or reduce disease transmission.

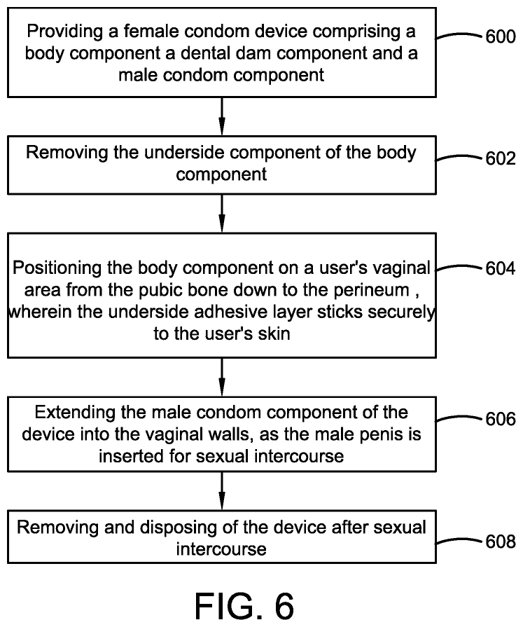

FIG. 6 illustrates a flowchart of the method of protecting a female user during sexual intercourse. The method includes the steps of at 600, providing a female condom device comprising a body component with a dental dam component and a male condom component. The method also comprises at 602, removing the underside adhesive layer of the body component. Further, the method comprises at 604, positioning the body component on a user's vaginal area from the pubic bone down to the perineum wherein the underside adhesive layer sticks securely to the user's skin. The method also comprises at 606, extending the male condom component of the device into the vaginal walls, as the male penis is inserted for sexual intercourse. Finally, the method comprises at 608, removing and disposing of the device after sexual intercourse.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “female condom device”, “condom device”, and “device” are interchangeable and refer to the female condom device 100 of the present invention.

Notwithstanding the foregoing, the female condom device 100 of the present invention can be of any suitable size and configuration as is known in the art without affecting the overall concept of the invention, provided that it accomplishes the above-stated objectives. One of ordinary skill in the art will appreciate that the female condom device 100 as shown in FIGS. 1-6 are for illustrative purposes only, and that many other sizes and shapes of the female condom device 100 are well within the scope of the present disclosure. Although the dimensions of the female condom device 100 are important design parameters for user convenience, the female condom device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A female condom device that protects a female from contraception and transmission of venereal diseases during sexual intercourse, the female condom device comprising:
a body component; and
an adhesive secured to an underside of the body component;
wherein the body component comprises a dental dam component and a male condom component; and
further wherein in use, the dental dam component remains outside a vagina of the female, while the male condom component extends into the vagina of the female.

2. The female condom device of claim 1, wherein the male condom device is formed of a generally cylindrical tube, with a distal end having an integrally formed rounded closure, and a proximal end that is open to allow for insertion of a male penis during sexual intercourse.

3. The female condom device of claim 2, wherein instead of a male penis, an applicator is inserted into the male condom component for purposes of insertion of the male condom component into the vagina of the female.

4. The female condom device of claim 3, wherein the female condom device is inserted prior to intercourse.

5. The female condom device of claim 4, wherein the proximal end of the male condom component is secured to the dental dam component.

6. The female condom device of claim 5, wherein the dental dam component includes a rectangular-shaped portion that flares outwardly away from the proximal end to cover the vagina of the female.

7. The female condom device of claim 6, wherein the dental dam component acts to cover a vaginal area and extends from a user's pubic bone down to a user's perineum.

8. The female condom device of claim 7, wherein the adhesive secures to a user's skin to keep the female condom device in place during sex and minimize a transfer of bodily fluids.

9. The female condom device of claim 8, wherein the adhesive can be an adhesive section, multiple adhesive sections, or an entire adhesive layer positioned on the underside of the dental dam component.

10. The female condom device of claim 9, wherein the adhesive is a strong, durable but comfortable, skin-safe adhesive.

11. The female condom device of claim 10, wherein the adhesive comprises a peelable protective strip, which is peeled off, exposing the adhesive when the female condom device is ready to be applied to the user.

12. The female condom device of claim 11, wherein the female condom device is easily removed by manually grasping the dental dam component and pulling the female condom device from the vagina.

13. The female condom device of claim 12, wherein the female condom device is produced in different lengths.

14. The female condom device of claim 13, wherein the male condom component comprises at least one pleat to accommodate a large penis.

15. A female condom device that protects a female from contraception and transmission of venereal diseases during sexual intercourse, the female condom device comprising:
a body component including a dental dam component and a male condom component; and
an adhesive secured to an underside of the body component;
wherein the male condom device is formed of a generally cylindrical tube, with a distal end having an integrally formed rounded closure, and a proximal end that is open to allow for insertion of a male penis during sexual intercourse;
wherein the proximal end of the male condom component is secured to the dental dam component;
wherein the dental dam component includes a rectangular-shaped portion that flares outwardly away from the proximal end to cover the user's vagina;
wherein the dental dam component acts to cover the vaginal area and extends from a user's pubic bone down to a user's perineum;
wherein the adhesive secures to a user's skin to keep the female condom device in place during sex, minimizing transfer of bodily fluids;
wherein in use, the dental dam component remains outside a user's vagina, while the male condom component extends into a user's vaginal walls;
wherein the adhesive comprises a peelable protective strip, which is peeled off, exposing the adhesive when the female condom device is ready to be applied to a user; and
further wherein the female condom device is easily removed by manually grasping the dental dam component and pulling the female condom device from the vagina.

16. The female condom device of claim 15 further comprising a plurality of indicia.

17. The female condom device of claim 15, wherein the female condom device is produced in different lengths.

18. The female condom device of claim 15, wherein instead of a male penis, an applicator is inserted into the male condom component for purposes of insertion of the male condom component into the user's vagina.

19. The female condom device of claim 15, wherein the adhesive is a strong, durable but comfortable, skin-safe adhesive.

20. A method of protecting a female user during sexual intercourse, the method comprising the following steps:
providing a female condom device comprising a body component with a dental dam component and a male condom component;
removing the underside adhesive layer of the body component;
positioning the body component on a user's vaginal area from the pubic bone down to the perineum, wherein the underside adhesive layer sticks securely to the user's skin;
extending the male condom component of the device into the vaginal walls, as the male penis is inserted for sexual intercourse; and
removing and disposing of the device after sexual intercourse.
